(19) United States
(12) Patent Application Publication
(10) Pub. No.: US 2008/0140021 A1
(43) Pub. Date: Jun. 12, 2008

(54) Spikeless Connection and Drip Chamber With Valve

(76) Inventor: Frank M. Richmond, Harvard, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/932,775

(22) Filed: Oct. 31, 2007

Related U.S. Application Data
(63) Continuation of application No. 11/040,886, filed on Jan. 21, 2005.
Application No. 11/040,886 is a continuation of application No. 08/768,636, filed on Dec. 18, 1996, now Pat. No. 5,848,994.
Application No. 08/768,636 is a continuation of application No. 08/612,875, filed on Mar. 12, 1996, now Pat. No. 5,645,538.
Application No. 08/612,875 is a continuation of application No. 08/123,632, filed on Sep. 16, 1993, now Pat. No. 5,405,333.
Application No. 08/123,632 is a continuation of application No. 08/751,310, filed on Nov. 18, 1996, now Pat. No. 5,735,826.
Application No. 08/751,310 is a continuation of application No. 08/377,514, filed on Jan. 24, 1995, now abandoned.
Application No. 08/377,514 is a division of application No. 08/098,499, filed on Jul. 28, 1993, now Pat. No. 5,445,623.

(51) Int. Cl. A61M 5/00

(52) U.S. Cl. 604/246; 604/247

(57) ABSTRACT

A drip chamber includes an elongated transparent container, and a cap. The container has open proximal and distal ends. The cap covers the distal end and further includes a drip forming tube, a cannula and an attachment element used to removably attach the chamber to a connector. The cannula extends distally away from the chamber and is surrounded by the attachment element. A pathway for fluid is established through the cannula and into the chamber.

### RELATED APPLICATIONS

The present application is a continuation of and claims priority from the following co-pending U.S. patent applications: U.S. patent application Ser. No. 11/040,886 for an invention entitled “Spikeless Connection and Drip Chamber with Valve”, filed Jan. 21, 2005, which in turn claims priority to U.S. patent application Ser. No. 08/768,636 for an invention entitled “IV Sets With Needleless/Spikeless Fittings And Valves”, filed Dec. 18, 1996, U.S. Pat. No. 5,645,538, filed on Mar. 12, 1996 for an invention entitled “Needleless Valve For Use In Intravenous Infusion”, which in turn claims priority from U.S. Pat. No. 5,405,333 filed on Sep. 16, 1993 for an invention entitled “Liquid Medicament Bag With Needleless Connector Fitting Using Boat Assembly.”

Additionally, this application claims priority from U.S. patent application Ser. No. 08/751,310 for an invention entitled “Drip Chamber With Female Luer Fitting” filed Nov. 18, 1996, which in turn claimed priority from Ser. No. 08/377,514 for an invention entitled “Drip Chamber With Female Luer Fitting” filed Jan. 24, 1995, which in turn is a divisional application of issued U.S. Pat. No. 5,445,623, issued on Aug. 29, 1995 for an invention entitled “Drip Chamber With Luer Fitting”. All are incorporated herein by reference.

### FIELD OF INVENTION

The present invention relates generally to intravenous (IV) liquid medicament infusion equipment, and more particularly to drip chambers, valves and attachment mechanisms.

### BACKGROUND OF THE INVENTION

One of the most widely used methods of medical therapy is the intravenous (IV) infusion of liquid medicaments and/or nutrients into the bloodstream of a patient. A familiar apparatus that is used in many IV infusion applications is an IV container, such as an IV bag or bottle, which contains the liquid to be infused into the patient.

When the IV container is a bag, or bottle, a rigid, hollow, sharpened IV spike is pushed into the container to establish a pathway for fluid communication through which the liquid can flow out of the container. The spike, in turn, is connected to or formed integrally with an inlet port of a small, elongated, transparent hollow container familiarly referred to as a “drip chamber”, with the fluid pathway of the spike in fluid communication with the inlet port of the drip chamber or by means of a port in a matching configuration to male or female connector with or without an integral valve.

Additionally, an IV line is connected to the bottom or distal end of the drip chamber. Preferably, a means for controlling the flow (a roller clamp, pump, or other suitable flow regulating device) is engaged with the IV line, and a medical technician can manipulate the flow controlling means and thereby regulate fluid flow through the IV line. To complete the path for fluid communication from the IV container to the patient, a sharp needle is connected to the IV line to puncture the patient.

Usually, the container is elevated above the patient to establish a positive pressure head to force the fluid that is within the container through the drip chamber into the patient. Because the drip chamber is transparent, a medical technician can view the medicament as it passes (normally by dripping) through the drip chamber to aid the medical technician in establishing a predetermined flow rate of medicament into the patient as the medical technician adjusts the flow controlling means on the IV line.

While effective as aiding in the establishment of a predetermined fluid flow to the patient, existing drip chambers, as noted above, require the use of sharpened spikes to puncture the IV container containing the liquid. This is undesirable, particularly in the era of AIDS, because spikes, like other sharps instruments, can inadvertently puncture the bag or medical technician who is manipulating the spike and thereby potentially contaminate the container contents or infect the technician with AIDS or other disease. Thus, as recognized by the present invention, it is desirable to avoid the use of sharp instruments whenever possible while preserving the quick connection such instruments provide.

Further, it is desirable to connect and disconnect the drip chamber or other components in the IV system without spillage of medicament. As recognized by the present invention, such reduction in spillage can be obtained through the use of reflex valves which are compatible with spikeless drip chambers and other needleless IV components.

Accordingly, it is an object of the present invention to provide a valve apparatus in an IV drip chamber or other IV component for engaging a complementary fitting, without the need to use a sharp connector. Another object of the present invention is to allow connection and disconnection of components without the spillage of medicament. Yet another object of the present invention to provide a drip chamber which is easy to use and cost-effective to manufacture.

### SUMMARY OF THE INVENTION

A drip chamber includes an elongated transparent container defining an elongated hollow chamber. The container has both a proximal end and a distal end. A cap covers the proximal end of the container, and it includes a drip-forming tube, a cannula and an attachment element. The drip forming tube is disposed within the container while the cannula extends distally away from the tube and establishes a pathway for fluid communication between a IV medicament container and the drip chamber. The attachment element surrounds the cannula and is configured so as to removably engage the chamber with a connector.

In one presently preferred embodiment, the distal end of the drip chamber is engageable with an IV tube and connector to establish a pathway for fluid communication between the drip chamber and a patient.

The cannula in the presently preferred embodiment, is metal, but the present invention recognizes that it may be formed from other materials such as plastic.

In another embodiment, the distal end of the container is a solvent bondable port element in fluid communication with the chamber. In yet another embodiment, the proximal and/or distal end of the container is also configurable as a luer fitting. The present invention recognizes that either a male or female luer fitting may be used in this embodiment.

On the proximal side of the container, to retain the chamber with an IV medicament connector, an attachment element is used. In one embodiment, the attachment element is configured as a threaded collar fitting. In another embodiment, the attachment element is configured as a so called “goal post style” clamp. The “goal post style” clamp has an open and a normal retention configuration, and is biased to the normal configuration. Preferably, the clamp includes two clamp elements, two fulcrum bars and two retaining lips. More clamp elements, fulcrum bars and lips are possible, but two of each is the most efficient. Specifically then, the clamp elements each have a proximal pincer end and a distal squeezeable end. The fulcrum bars are then attached on one side to the clamp element and on the opposite side to the cannula element or cannula holding element. The fulcrum bars are long enough such that the proximal pincer ends are separated when the clamp is in the normal configuration. Ideally, the pincer ends open to facilitate easy assembly of the connector. The proximal pincer ends may also be configured with a lip to engage a complementary surface on the connector. The connector can be the port of any IV device, but the port of an IV bag, or other source of fluid, is preferable. To further facilitate engagement of the “goal post style” clamp, the lips include an angular surface which, when urged against the connector port, move the pincer ends open sufficiently to allow mating of the lip and the complementary connector surface.

In another embodiment, a drip chamber includes an elongated container defining a hollow chamber. The chamber has both a proximal end and a distal end. A cap preferably covers the proximal end of the container and it includes a drip forming tube, a valve body, at least one valve member disposed in the valve body and a valve actuating element. The drip forming tube is disposed within the container while the valve body defines a pathway for fluid communication through the cap. The valve member is disposed in the body and is biased to a first configuration where the path for fluid communication is not established. That is, in the first configuration, fluid may not pass through the body. Additionally, the valve member is movable to a second configuration where fluid communication through the body is permitted. Also disposed in the valve body is the valve actuating element. This element defines at least one engagement surface for contacting a mating element. Contact with the mating element causes the valve actuating element to move against the valve member which causes the valve member to move to the second configuration.

A variation of the above includes a valve member defining an outer periphery that is interrupted at least once within the periphery. The interruption within the periphery allows the fluid to pass directly through the member when in the second configuration rather than around the member (although the fluid could also pass through and around the member as envisioned above). In other words, when the valve member is in the second configuration, the interruption within the periphery defines an opening in the valve member allowing fluid through the valve member and thus, through the body.

In another variation, valves that may be utilized include valves that include a resilient valve member. In this aspect, the resilient valve member defines an outer periphery that is uninterrupted within the periphery. The valve member is deformable to a configuration wherein fluid communication is permitted.

The proximal and distal ends of the drip chamber may be configured in a variety of ways. In one embodiment the proximal or distal end is configured as a male or male luer fitting. In another the proximal or distal end is configured with a solvent bonded IV tube. And in yet another the proximal or distal end is configured as a female or female luer fitting.

In another preferred embodiment, an IV component connector includes a valve body, a valve member and a valve element. In this embodiment, the valve body has a proximal and a distal end which define a path providing fluid communication through the body. The proximal end has an attachment element to engage a complementarily shaped connector or surface. The distal end has an outlet providing fluid communication with a connected component.

The valve member in this embodiment is disposed in the body and defines an outer periphery that is interrupted within the periphery at least once. The valve member is biased to a first configuration where the path for fluid communication is not established through the body. Also, the member is movable to a second configuration where fluid communication is permitted. The valve element is also disposed in the body and defines at least one engagement surface distally beyond the body. This surface contacts a mating element from another component that causes the valve element to move against the valve member thus moving the valve member to the second configuration.

In one presently preferred embodiment, the attachment element is configured as a male or male luer fitting to removably engage a female or female luer connector.

In another variation, the component connected to the proximal end may be any of various other IV components as disclosed in U.S. Pat. No. 5,645,538 to Richmond and incorporated herein by reference. Particular attention is directed to FIGS. 13, 15, 18, 19, 22-30; and the Specification, column 2, lines 14-18.

Another aspect of the invention is directed to a drip-chamber that includes a cap and a drip forming tube with a blunt cannula integrally attached to the cap and drip forming tub. The drip-chamber includes an additional means of attachment which is integrally attached to the cap.

These and other aspects of the present invention can best be appreciated in reference to the accompanying drawings in which like numerals refer to like parts, and in which:

### BRIEF DESCRIPTION OF THE DRAWINGS

FIG. 1 is a cross-sectional view of a valve of the present invention showing a male cannula fitting drip chamber combined with a goal post style clamp;

FIG. 2 is a cross-sectional view of an alternate inserted male cannula fitting drip chamber combined with a goal post style clamp;

FIG. 3 is a cross-sectional view of a drip chamber with an embedded cannula fitting combined with a threaded collar fitting;

FIG. 4 is a cross-sectional view of a drip chamber with a cannula combined with a threaded collar fitting;

FIG. 5 is a cross-sectional view of one embodiment of the IV component connector;

FIG. 6 is a cross-sectional view of another embodiment of the IV component connector;

FIG. 7 is a cross-sectional view of yet another embodiment of the IV component connector;

FIG. 8 is a cross-sectional view of the connector as shown in FIG. 5 combined with a drip chamber and a distal end configured as a male luer fitting;

FIG. 9 is a cross-sectional view of the connector as shown in FIG. 6 combined with a drip chamber and a distal end configured as a solvent bonded IV tube;

FIG. 10 is a cross-sectional view of the connector as shown in FIG. 7 combined with a drip chamber and a proximal end configured as a female luer fitting.

FIG. 11 is a partial cross-sectional view of an IV set of the present invention, showing various drip chamber upper connections and various drip chamber lower connections in exploded relationships.

FIG. 12 is a cross-sectional view of a male reflux valve bonded to a “T”-site connector.

FIG. 13 is a cross-sectional view of a male reflux valve bonded to a “Y”-site connector.

FIG. 14 is a plan cross-sectional view of a plurality of reflux valves operably engaged with a stopcock.

FIG. 15 is an exploded cross-sectional view of various reflux valves in combination with a filter assembly.

FIG. 16 is an exploded isometric view of a four-way valve with various associated components including luer fittings with reflux valves.

FIG. 17 is a cross-sectional view of a male luer fitting with male reflux valve in combination with a check valve.

FIG. 18 is an exploded cross-sectional view of another alternate embodiment of the liquid medicament bag with portions broken away showing a valve connected to a medicament bag by a tubular connector.

FIG. 19 is a cross-sectional view of a male reflux valve shown disposed in a male luer fitting, with portions broken away for clarity.

FIG. 20A is a view of one of the valves of the IV bag with the valve in the closed position.

FIG. 20B is a view of one of the valves of the IV bag with the valve in the open position.

FIG. 21 is a perspective view of a liquid medicament bag having a male and a female reflex valve, with portions of the bag broken away.

FIG. 22 is a cross-sectional view of the liquid medicament bag of the present invention, as seen along the line 6-6 in FIG. 21.

FIG. 23 is a cross-sectional view of the liquid medicament bag of the present invention, as seen along the line 7-7 in FIG. 21, with the valve in the closed configuration.

FIG. 24 is a cross-sectional view of the liquid medicament bag of the present invention, as would be seen along the line 7-7 in FIG. 21, with a female luer fitting shown in phantom engaging the valve, with the valve in the open configuration;

### DETAILED DESCRIPTION OF THE PREFERRED EMBODIMENT

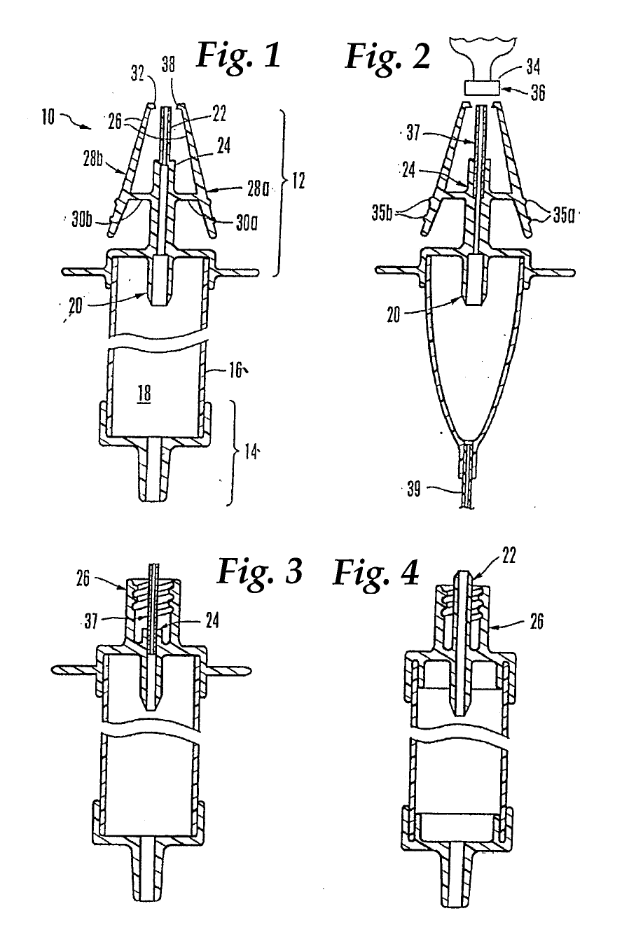

Referring initially to FIG. 1, the drip chamber with cannula of the present invention is shown and is generally designated as 10. Preferably, the drip chamber 10 is made of a plastic (e.g. polypropylene, polyethylene, etc.) or glass or metal. As shown, the drip chamber 10 has a proximal end 12 which can be engaged with a source of fluid, such as an IV container (not shown). It is to be understood that the source of fluid can be any container suitable for holding fluid medicaments, e.g., the source can be an IV bag, vial, IV bottle, semi-rigid container, syringe, etc.

As further shown in FIG. 1, the drip chamber 10 has a distal end 14 that can be engaged with an IV tube (shown as 39 in FIG. 2). Specifically, the IV tube 39 is advanced onto the distal end 14 of the drip chamber 10 and is held on the distal end 14 by solvent bonding, RF sealing, ultrasonic welding techniques, or other techniques known by those skilled in the arts.

Still referring to FIG. 1, the drip chamber 10 includes a hollow transparent glass or plastic container 16, and the container 16 defines a hollow chamber 18. As shown, the proximal end 12 is configured as a cap including a drip forming tube 20, a cannula 22 formed integrally with a cannula holding element 24 and an attaching mechanism generally designated 26.

Cross referencing FIGS. 1 and 2, the attaching mechanism 26 is a goal post style clamp. As shown, the goal post style clamp consists of two clamp elements 28a, 28b. The clamp elements 28a, 28b are attached to the cannula holding element 24 by two fulcrum bars 30a, 30b. It can be noted now that the goal post style clamp is biased to the retention configuration shown in the FIGS. The clamp elements 28a, 28b each have a lip 32 on the distal pincer end to engage a ledge 34 on the fluid source connector port 36. The lip 32 is configured with an angular surface 38 to facilitate engagement of the connector port 36. The distal end of the clamp elements 28a, 28b can have bumps 35a, 35b to improve the operator's grip while applying pressure to the clamp elements 28a, 28b. Squeezing pressure on the clamp elements 28a, 28b, below the fulcrum bars 30a, 30b, urges the clamp elements 28a, 28b, to an open configuration.

FIG. 2 shows an alternate embodiment which is identical to FIG. 1 in all essential respects, except that the cannula 37 is embedded into the cannula holding element 24. Additionally, FIG. 2 also shows the IV tube 39 inserted into the distal end 14 of the drip chamber 10 and held in the distal end 14 by solvent bonding, RF sealing, ultrasonic welding techniques, or other techniques known by those skilled in the arts.

FIG. 3 shows an alternate embodiment which is identical in all essential respects to FIG. 2 except that the attaching mechanism 26 is alternately configured as a threaded collar surrounding the embedded cannula 37.

FIG. 4 shows an alternate embodiment which is identical in all essential respects to FIG. 1 except that the attaching mechanism 26 is configured as a threaded collar surrounding the integral cannula 22.

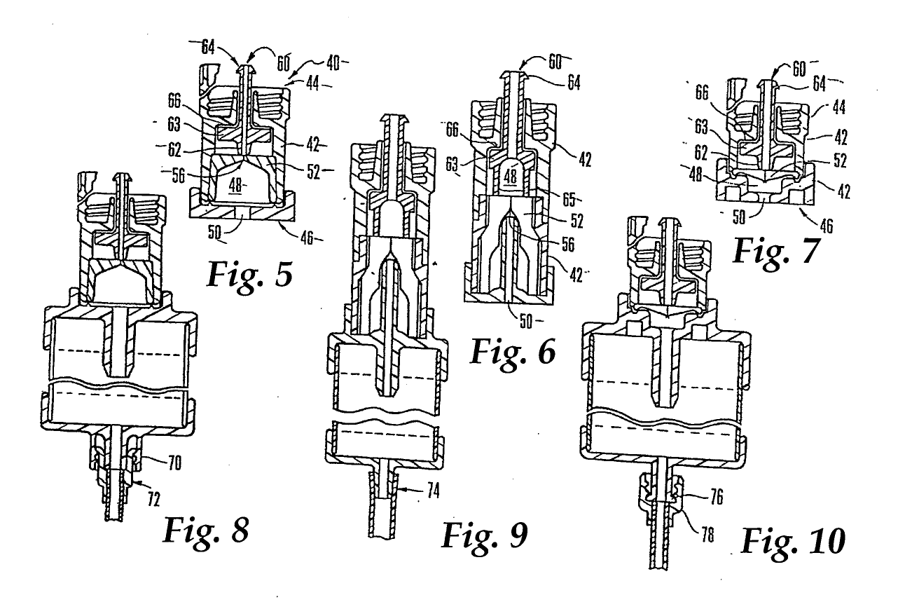

Now referring to FIG. 5, an IV component connector is shown and generally designated as 40. The connector 40 has a valve body 42 which has a proximal end 44 and a distal end 46. The body defines a fluid passageway 48 which provides a pathway for fluid communication through the body 42. As can be readily observed in FIG. 5, the proximal end 44 is configured as a male fitting for connection to a corresponding female fitting (not shown). Fluid communication through the body 42 ends at the distal end 46 where an outlet 50 is provided. The distal end 46 attaches to various other IV components as disclosed in issued U.S. Pat. No. 5,645,538 to Richmond and incorporated herein by reference.

Still referring to FIG. 5, a valve member 52 is shown disposed in the body 42 to selectively block the fluid passageway 48. The valve member 52 is preferably made from plastic, silicone, rubber, etc., and defines an outer periphery that may be interrupted by a fluid orifice 56. Those skilled in the art will recognize that more than one fluid orifice 56 is possible and that the orifice 56 may be shaped in a variety of ways. When the valve is in place in a closed configuration, the orifice 56 is sealed and consequentially, a fluid tight seal between the valve member 52 and the interior surface of the body 42 is established. It is to be understood that the valve member 52 is biased to the closed configuration as shown in FIG. 5, wherein no fluid communication is permitted through the valve body 42 (and hence through the outlet 50). On the other hand, when pressure is exerted on the member 52 from the proximal side 44 of the valve member 52, the member 52 is moved to cause the fluid orifice 56 to open and, thus, to move the member 52 to the open configuration.

FIG. 5 additionally shows that a valve element 60 is reciprocably disposed in the fluid passageway 48. The valve element 60 is formed with a lower probe 62, a retention element 63 and an upper contact flange 64. When the valve element 60 is urged into the valve body 42, the lower probe 62 exerts pressure, thus opening the valve member 52 as discussed above. As shown, the retention element 63 retains the valve element 60 in the valve body 42 by contacting a surface 66.

Still referring to FIG. 5, it can now be understood that the proximal end 44, configured as a male luer fitting, can be engaged with a complementarily shaped female luer fitting (not shown). This engagement causes the upper contact flange 64 of the valve element 60 to be contacted by the female luer fitting (not shown) and to urge the valve element 60 into the valve body 42. When the valve element 60 is urged sufficiently, it contacts the valve member 52 and urges the valve member 52 to the open configuration, thereby allowing fluid communication through the orifice 56, and hence through the fluid passageway 48.

Now referring to FIG. 6, an alternate embodiment of the IV component connector is shown. This is in all essential respects identical to FIG. 5, except that the valve element 60 has a skirt 65 for urging the valve member 52 downwardly and further urging the fluid orifice 56 to the open configuration. Also, as can best be seen in FIG. 6, the valve member 52 need not be rigidly attached to the valve body 42.

FIG. 7 shows yet another alternative embodiment of the IV component connector of the present invention. This is in all essential respects identical to FIG. 5, except that the valve member 52 can also be held in place by trapping between the separate sub-components of the valve body 42. This Figure also best illustrates an alternate valve member 52. In this embodiment, the distal side of the valve member 52 is essentially flat while the proximal side bulges in the center. Said another way, the thickness of the valve member 52 increases towards the center. Importantly, because of the thickening of the valve member 52 towards the center on the proximal side of the valve member 52, pressure from the proximal side 46 of the valve member 52 will cause the valve member 52 to close more tightly. That is, backflow pressure will act to dose the valve member 52 and increased pressure will more tightly close the valve 52.

FIGS. 8, 9 and 10 show the various embodiments of the component connector of the present invention in combination with drip chambers. FIG. 8 also shows the distal end configured as a male luer fitting 70. The male luer fitting 70 can then be connected and disconnected from a corresponding female fitting 72. FIG. 9 shows the distal end configured as a solvent bonded IV tube 74. FIG. 10 shows the distal end configured as a female luer fitting 76. The female luer fitting 76 can then be connected and disconnected from a corresponding male fitting 78.

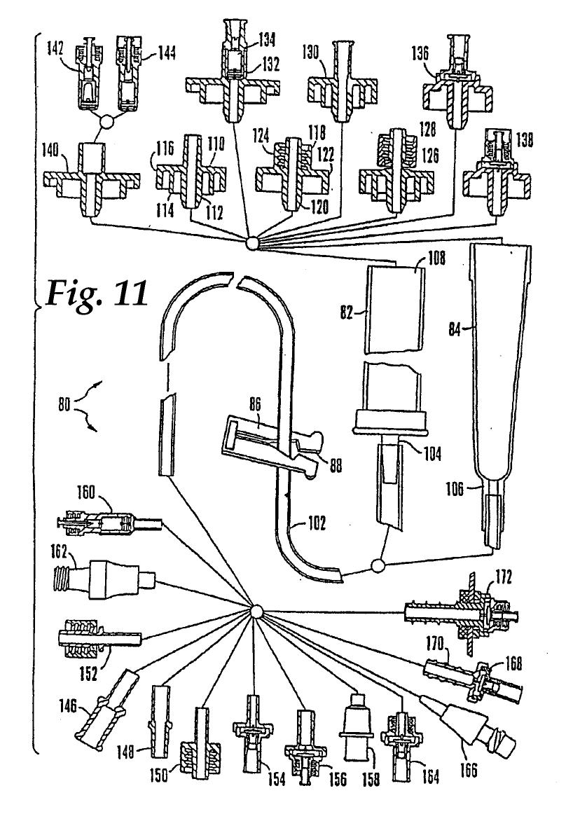

Now referring to FIG. 11, an IV set is shown, generally designated 80. As can be appreciated in reference to FIG. 11, the present IV set includes a drip chamber, an upper needleless connector on the top of the drip chamber to connect the top to a needleless fitting (such as any of those shown herein), an IV tube connected to the bottom of the drip chamber, a flow restrictor engaged with the IV tube, and a lower needleless connector connected to the end of the IV tube to connect the tube to a needleless fitting (such as any of those shown herein).

As shown in FIG. 11, the drip chamber can be an elongated hollow transparent plastic cylindrical drip chamber 82 or tapered drip chamber 84. In any case, the present drip chamber is “elongated” in that its length is at least half again as great as its diameter. The IV tube is a hollow plastic IV tube known in the art, with the flow restrictor being an open slide clamp 86 having an open head end 88 (FIG. 11).

in the particular embodiment shown in FIG. 11, an IV tube 102 can be connected by attaching the tube 102 by means well-known known in the art to an exit port 104 of the cylindrical drip chamber 82 or to and exit port 106 of the tapered drip chamber 84. For succinctness of disclosure, the discussion below will focus on the cylindrical drip chamber 82, but it is to be understood that the discussion below is equally relevant to the tapered drip chamber 84 or indeed any well-known drip chamber. The open clamp 86 can be manipulated by means well-known in the art to constrict the IV tube 102 to stop fluid flow therethrough.

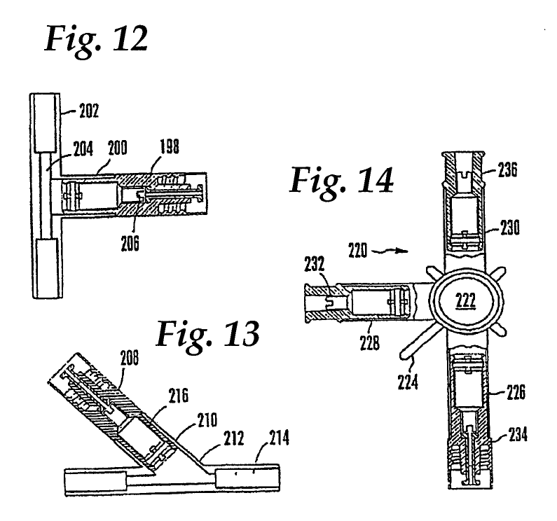

FIG. 12 shows a male member valve 198 which is disposed in a port 200 of a so-called “T”-site connector 202. The T-site connector 202 defines a main fluid passageway 204 and a secondary fluid passageway 206, and the male valve 198 can be manipulated as described above to selectively permit fluid communication through the secondary fluid passageway 206 of the T-site connector.

FIG. 13 shows a male member valve 208 which is disposed in a port 210 of a so-called “Y”-site connector 212. As shown, the Y-site connector 212 defines a main cylindrical fluid passageway 214 and a secondary fluid passageway 216. The valve 208 can be operated as disclosed above to selectively block fluid communication through the secondary passageway 216 of the Y-site connector 212.

FIG. 14 shows an IV stopcock, generally designated 220. In accordance with principles well known in the art, the stopcock 220 includes a central fluid passageway that is covered by a cover plate 222, and an operating hand wheel 224. Additionally, the stopcock 220 can include at least two ports, and can include additional ports.

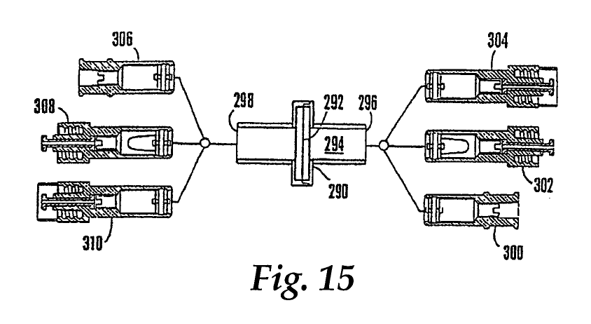

As shown in FIG. 15, the IV component can be a tubular IV connector 290 having a filter 292 disposed athwart a fluid passageway 294 defined by the connector 290. The connector 290 has an inlet port 296 and an outlet port 298. The inlet port 296 can be selectively blocked by engaging the port 296 with any one of a female member valve 300, a first male member valve 302, or a second male member valve 304. Similarly, the outlet port 298 can be selectively blocked by engaging the port 298 with any one of a female member valve 306, a first male member valve 308, or a second male member valve 310. Accordingly, the IV component shown in FIG. 15 is resealable, in that upon disconnecting a fitting from the valve member in one of the ports 296, 298, fluid flow through the component is prevented by the valve member in the disconnected port.

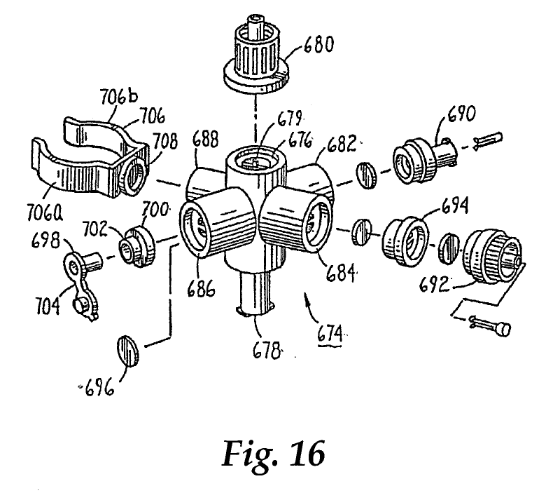

Now referring to FIG. 16, a novel multiport IV valve of the present invention, generally designated 674, can be seen. As shown, the multiport valve 674 includes a first port 676 which is generally cylindrically shaped, and a second port 678 which is also generally cylindrically shaped and is configured as a female Luer fitting.

FIG. 16, the first and second ports 676, 678 are coaxial and establish a main fluid passageway therebetween. A first male luer valve 680 is fixedly engaged with the first port 676 for selectively blocking fluid communication therethrough.

FIG. 16 additionally shows that the multiport valve 674 includes third, fourth, fifth and sixth ports 682, 684, 686, 688, all of which are generally cylindrically shaped. As shown, the third and fifth ports 682, 686 are coaxial with each other. Likewise, the fourth and sixth ports 684, 688 are coaxial with each other. Each of the third through sixth ports 682, 684, 686, 688 defines a respective fluid pathway, and fluid communication through the fluid pathway can be selectively established or otherwise effected as disclosed below.

Still referring to FIG. 16, for example, a female reflux valve 690 can be disposed in the third fluid port 682 for selectively establishing fluid communication through the port 682 and into the main fluid passageway 679 in accordance with principles disclosed previously. Moreover, a combination male reflux valve-check valve 692, 694, can be disposed in the fourth fluid inlet port 684 of the multiport valve 674. If desired, the male reflux valve 692 can be replaced with a female reflux valve (not shown) which is substantially identical to the female reflux valve 308 and/or 310 shown in FIG. 15.

Still referring to FIG. 16, additionally, fluid communication through the fifth inlet port 686 can be permanently blocked if desired by bonding a plug 696 within the port 686 by means well known in the art. Alternatively, the fifth fluid inlet port 686 can hold a fluid filter, e.g. a filter 698. As shown, the filter 698 includes a filter element 700 having a membrane 702 through which air can pass. The filter 698 also includes a plug element 704 which engages filter element 700 and which holds the filter element 700 within the sixth fluid port 686.

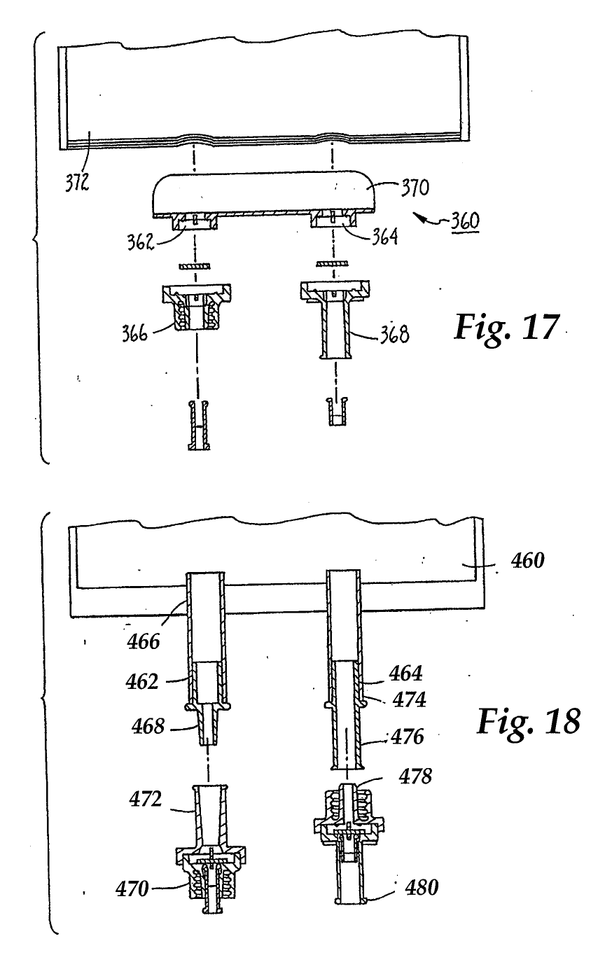

FIG. 17 shows a valve assembly, generally designated 360, which has a so-called boat shape, and is accordingly referred to in the art familiarly as a “boat”. The assembly 360 also has two openings 362, 364 and two valves 366, 368 respectively positioned in the openings 362, 364. The valves 366, 368 can be female or male valves.

As can be appreciated in reference to FIG. 17, the assembly 360 has a hull surface 370 which is generally shaped like the gently rounded hull of a boat, and which consequently can be positioned between the juncture of two sides of a bag 372, and then attached to the bag by means well-known in the art. In other words, the hull surface 370 of the assembly 360 can be positioned in the seam of a liquid medicament bag.

In addition to the openings discussed above, the assemblies 308, 370, 460 can have additional openings (not shown) which can be blocked by respective reflex valves or by pierceable membranes.

FIGS. 17 and 18 show multi-layer medicament bags having multiple individual tubular connectors for receiving associated valves. More specifically, FIG. 18 shows a bag 460 which has a first tubular connector 462 and a second tubular connector 464. The first tubular connector 462 includes a hollow cylindrical tube 466 which is bonded to the bag 460 by means well-known in the art.

Now referring to FIG. 18, a hollow male luer connector 468 is closely received in the tube 466, and a female luer fitting 472 can be engaged with the male luer connector 468. A valve 470 may be bonded to the female luer fitting 472.

Still referring to FIG. 18, likewise, the second tubular connector 464 includes a hollow cylindrical tube 474 which is bonded to the bag 460 by means well-known in the art. A female luer connector 476 is closely received in the tube 474, and a male luer fitting 478 can be engaged with the female luer connector 476. A valve 480 may be bonded to the male luer fitting 478. IV lines or other components, e.g., spikes and drip chambers (not shown) having luer fittings can be engaged with the valves 470, 480 to selectively establish fluid flow through the connectors 462, 464 in accordance with the principles set forth above.

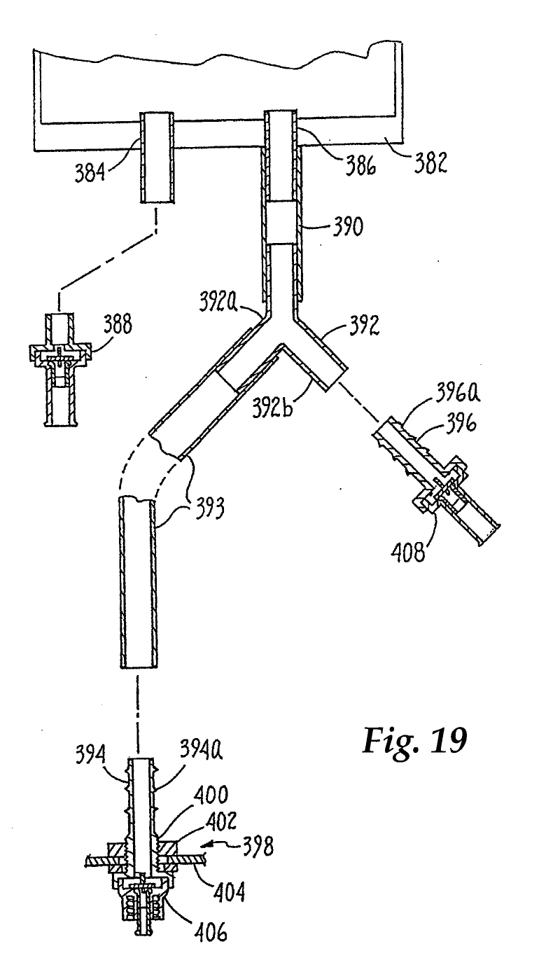

FIG. 19 shows that a multi-layer medicament bag 382 has a plurality of resilient plastic tubular connectors 384, 386. The connector 384 can closely receive a valve 388. If desired, the valve 388 can be bonded to the connector 384.

FIG. 19 also shows that an intermediate connector tube 390 can be bonded to the connector 386, and a hollow Y-fitting 392 in turn bonded to the intermediate connector tube 390. If desired, an extender tube 393 can be connected to a first leg tube 392a of the Y-fitting 392, and a first luer bulkhead fitting 394, can be advanced into the extender tube 393. A second luer bulkhead fitting 396 can be advanced into a second leg tube 392b of the Y-fitting 392 or extension tubes (not shown) that are connected to the second leg tube 392b.

Each of the luer bulkhead fittings 394, 396 has respective ratchet rings 394a, 396a which are configured as shown for permitting the luer bulkhead fittings 394, 396 to be advanced into their respective tubes 393, 392b, and for preventing the luer bulkhead fittings 394, 396 from being easily retracted from their respective tubes 393, 392b. If desired, the luer bulkhead fitting 394 can also include a locking ring assembly, generally designated 398, which includes a threaded body 400, a locking ring 402 threadably engaged with the body 400, and a backing ring 404. The luer bulkhead fittings 394, 396 can advantageously be similar to any one of the luer bulkhead fittings made by Value Plastics, Inc. of Fort Collins, Colo.

A male reflex valve 406 may be bonded by means well-known in the art to the first bulkhead luer fitting 394. Likewise, a female reflex valve 408 may be bonded by means well-known in the art to the second bulkhead luer fitting 396. IV lines or other components, e.g., spikes and drip chambers (not shown) having luer fittings can be engaged with the valves 388, 406, 408 to selectively establish fluid flow through the connector 384 and Y-leg tubes 392a, 392b (and extension tubes thereof e.g., the tube 393) in accordance with the principles set forth above.

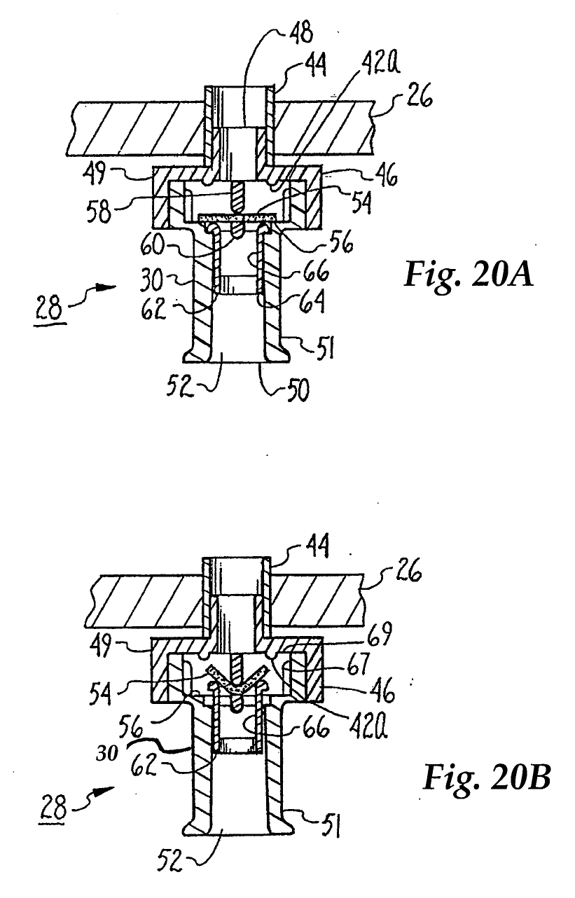

Now referring to FIG. 20A, the details of the spikeless/needleless port 28 can be seen. As shown, the spikeless/needleless port 28 includes a hollow tube 44, and the valve body 30 is positioned in the tube 44 to selectively prevent fluid communication through the tube 44. The valve body 30 includes a rigid, preferably plastic (e.g., PVC, etc.) valve body 46 that has a fluid inlet 48, a fluid outlet 50, and a fluid passageway 52 formed in the valve body 46 between the inlet 48 and outlet 50. The valve body 46 can be a unitary structure, or be made of two or more pieces that are bonded together, as shown. For example, the inlet 48 can be formed from a first piece 49, the outlet 50 can be formed from a second piece 51, and the two pieces can be bonded together by means well-known in the art, e.g., solvent bonding, ultrasonic sealing, or RF welding.

In cross-reference to FIG. 20A, the valve body 30 also includes a flexible resilient plastic or silicon rubber valve 54 that is disposed in the fluid passageway 52. Specifically, the periphery of the valve 54 rests on a seating surface 56 of the valve body 46 to establish a fluid-fight seal between the valve 54 and seating surface 56. In other words, the valve 54 is biased to the closed configuration shown in FIG. 20A. A support element 58 is formed in the fluid passageway 52 and extends across the fluid passageway 52.

Still referring to FIG. 20A, the support element 58 supports the valve 54 in the center thereof. To this end, a slight depression may be formed in the center of the valve 54 to receive the support element 58 and thereby prevent side-to-side motion of the valve 54 relative to the support element 58. As shown, the support element 58 is shaped as a cylinder, but it is to be understood that the support element 58 can have other suitable shapes, e.g., the support element 58 can have a triangular shape.

Additionally, in FIG. 20A, a retainer element 60 is formed on the valve body 46 and extends across the fluid passageway 52. As shown, the retainer element 60 is positioned on the valve body 46 on the opposite side of the valve 54 from the support element 58. Accordingly, the retainer element 60 holds the center of the valve 54 against the support element 58.

Still referring to FIG. 20A, a rigid urging member 62 is shown slidably disposed in the fluid passageway 52 for reciprocal movement therein. As shown, the urging member 62 has an annular head 64 and a skirt 66 that depends from the head 64. As further shown, the skirt 66 includes a plurality of, preferably two, legs. The urging member 62 can be forced against the valve 54 by advancing an appropriate connector fitting (not shown), such as a male luer fitting, into the fluid passageway 52 and against the urging member 62.

As shown in FIG. 20B, when the urging member 62 is forced against the valve 54, the skirt 66 of the urging member 62 contacts the surface of the valve 54. This deforms the valve 54, causing the sealing surface of the 54 to be distanced from the seating surface 56 of the valve body 46, and thereby permitting fluid communication through the fluid passageway 52. Stated differently, a spikeless/needleless connector can be advanced into the fluid passageway 52 to force the urging member 62 against the valve disc 54 and deform the disc 54 into an open configuration. When the spikeless/needleless connector is retracted from the fluid passageway 52, the resiliency of the valve 54 causes the valve 54 to resume its normally dosed configuration, shown in FIG. 20A.

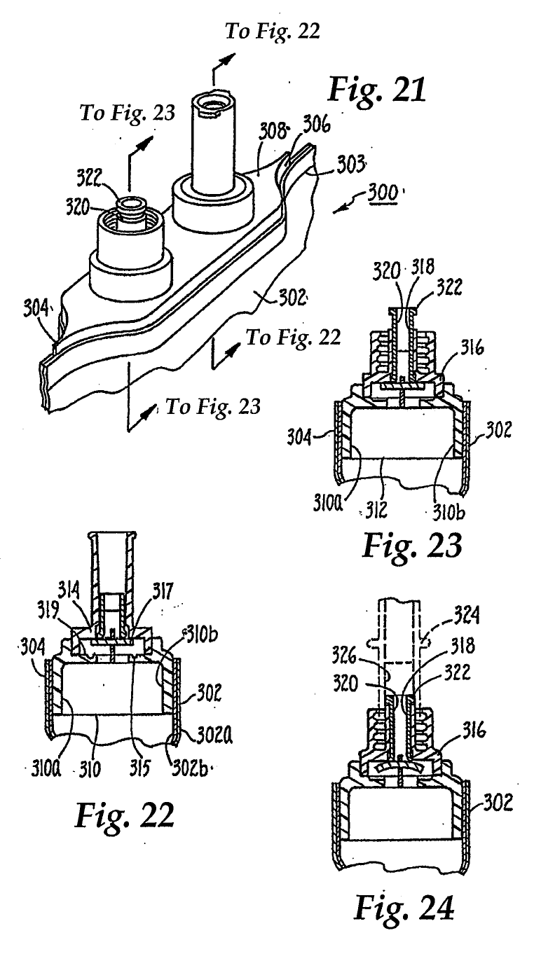

FIG. 21 shows that a liquid medicament bag 300 has a first multi-laminate side 302, a second multilaminate side 304, and a seam 306 that is established at the juncture of the sides 302, 304. Thus, the sides 302, 304 with seam 306 establish a container of the type well-known in the art for holding, e.g., liquid medicament, cell culture, and other biotech fluids. The skilled artisan will accordingly appreciate that the material and thickness of the laminated layers are selected for strength, puncture resistance, gas permeability, and compatibility with the fluid contained in the bag 300. For example, FIG. 22 shows that the side 302 has two layers 302a, 302b. It is to be appreciated that the side 302 can have additional layers, if desired.

Referring to FIG. 21, a rigid plastic valve assembly 308 is fixedly positioned on the seam 306 during manufacture by means well-known in the art, e.g., by RF sealing, spin welding, or ultrasonic welding, and the sides 302, 304 of the bag 300 are likewise connected, as indicated by a bond line 303. The assembly 308 in the perspective view shown has a catenary shape when looked at from above.

FIGS. 22 and 23 show that the valve assembly 308 is “H”-shaped in transverse cross-section, and has first and second openings 310, 312 which are established by flanges 310a, 310b. As shown, the flanges 310a, 310 bare bonded to the sides 304, 302, respectively, of the bag 300. A first reflex valve 314 is positioned in the first opening 310 and a second reflex valve 316 is positioned in the second opening 312.

As shown in FIG. 22, the first valve 314 is in all essential respects identical in construction to the valve 30 disclosed above, except that the first valve 314 has grooves 315 formed in place of the protrusions 42a shown in FIGS. 20A and 20B. It is to be understood that the grooves 315 fulfill the same function as the protrusions 42a, i.e., the grooves 315 prevent a vacuum lock from being established between a valve 317 and a valve body 319.

As shown best in FIG. 23, the second valve 316 also is identical to the valve 30, except that the second valve 316 has a male valve element 318. More particularly, the male valve element 318 has a cylindrical skirt 320 and a contact surface, preferably a disc-shaped head 322 which extends radially outwardly from the skirt 320. Alternatively, the valve element 318 can have a non-rounded shape.

As shown, the second valve 316 is configured as a male luer fitting. Consequently, a female luer fitting 324 (FIG. 24) can be engaged with the valve 316 such that a tapered inner surface 326 of the female luer fitting 324 urges against the head 322 to move the second valve 316 to the open configuration.

While FIGS. 22 and 23 show a valve assembly 308 having two valves 314, 316 positioned therein, it is to be understood that the assembly 308 can have one or more openings, and that one or more of the openings can have a piercable membrane positioned therein, if desired. It is to be further understood that the male valve 316 can be used in place of the valves previously disclosed, e.g., the male valve 316 can be used in place of the valve 30 shown in FIGS. 20A and 20B.

While the particular drip chamber with valve as herein shown and described in detail is fully capable of attaining the objects stated above, it is to be understood that it is but the presently preferred embodiments of the present invention, and that the scope of the present invention is accordingly to be limited by nothing other than the appended claims where singular nouns do not mean “one and only one,” but rather, “at least one” unless otherwise specifically noted as “one and only one.”

What is claimed is:

1. A drip-chamber comprising:
a cap and drip forming tube with a blunt cannula, wherein the blunt cannula and drip forming tube are an integral part of the cap; and
an additional means of attachment which is integral part of the cap.

2. The drip-chamber of claim 1 configured with a set.

3. The drip-chamber of claim 2, wherein the set further comprises a container with at least one port, wherein the port on the container is configured with a means of attachment.

4. The drip-chamber of claim 1, wherein the cannula is manufactured from plastic or metal.

5. The drip-chamber of claim 1, wherein the cannula is configured with straight parallel walls.

6. The drip-chamber of claim 1, wherein the cannula is configured with at least one tapered wall.

7. The drip-chamber of claim 3, wherein the drip-chamber configured with a cannula is connected to the port on the container.

8. The drip-chamber of claim 3, wherein at least one port on the container is configured with a means of attachment and the port is open.

9. A drip-chamber set comprising:
a drip-chamber with a means of attachment integrally attached to the drip-chamber; and a container connected to the drip-chamber, the container including at least one port configured with a means of attachment.

10. The drip-chamber set of claim 9, wherein the means of attachment on the drip-chamber includes a reflux valve.

11. The drip-chamber of claim 9, wherein the container is a semi-rigid, flexible, glass, plastic, metal, multi-layered construction or any combination thereof.

12. The drip-chamber of claim 9, wherein the means of attachment on the container is selected from the group consisting of a thread, a partial thread, a collar with a thread, ears, an annular protrusion and a partial annular protrusion.

13. The drip-chamber of claim 9, wherein the means of attachment on the container includes a reflux valve.

14. The drip-chamber set of claim 9, wherein the port configured with the means of attachment is open.

15. A medical device including at least one female reflux valve and at least one other component for conveying or containing liquid medicament, the or each valve comprising: a valve body having a distal end and a proximal end, the body defining a fluid passageway providing fluid communication through the body; a valve member disposed in the body, the valve member being biased to a first position wherein the fluid communication is not established through the body, the member being moveable to a second position, wherein two-way fluid communication through the body is permitted; and a valve element disposed in the passageway, entirely inside the valve body, in direct contact with the valve member for displacement within the passageway, the valve element having an engagement surface for contact with a spikeless/needleless connector for displacing the valve element to cause the valve member to move to the second position, wherein the component is selected from the group consisting of filters, burette chambers, stopcocks, connector fittings, vials, check valves, spikes, containers and multiport valves.

16. A medical device including at least one female reflux valve and at least one other component for conveying or containing liquid medicament, the or each valve comprising: a valve body having a distal end and a proximal end, the body defining a fluid passageway providing fluid communication through the body; a valve member disposed in the body, the valve member being biased to a first position wherein the fluid communication is not established through the body, the member being moveable to a second position, wherein two-way fluid communication through the body is permitted; and a valve element disposed in the passageway, entirely inside the valve body, in direct contact with the valve member for displacement within the passageway, the valve element having an engagement surface for contact with a spikeless/needleless connector for displacing the valve element to cause the valve member to move to the second position, wherein the component is a two-way valve requiring a female valve element for opening the two-way valve constituting the component.

17. A medical device including at least one female reflux valve and at least one other component for conveying or containing liquid medicament, the or each valve comprising: a valve body having a distal end and a proximal end, the body defining a fluid passageway providing fluid communication through the body; a valve member disposed in the body, the valve member being biased to a first position wherein the fluid communication is not established through the body, the member being moveable to a second position, wherein two-way fluid communication through the body is permitted; and a valve element disposed in the passageway, entirely inside the valve body, in direct contact with the valve member for displacement within the passageway, the valve element having an engagement surface for contact with a spikeless/needleless connector for displacing the valve element to cause the valve member to move to the second position, wherein the component is a two-way valve requiring a male valve element for opening the two-way valve constituting the component.

18. A device according to claim 16 wherein there is an intermediate structure.

19. A device according to claim 17 wherein there is an intermediate structure.

20. A device according to claim 19 wherein the intermediate structure is selected from the group consisting of containers, fluid lines, sets, drip chambers, filters, burette chambers, stopcocks, multi-port valves, ‘y’-sites and ‘t’-sites.

21. A device according to claim 19 having at least one distal end and at least one proximal end, wherein the two-way valve constituting said component is on at least one of the proximal and distal ends of the medical device.

22. A device according to claim 21 in which all distal and proximal ends have a reflux valve effective for providing resealable components or systems.

23. A device according to claim 21 wherein the reflux valve, on the other of the proximal and distal ends of the device, is female.

24. A device according to claim 15 in which one of the multiport valves includes an attachment feature.

25. A device according to claim 15 in which on of the multiport valve includes a filter.
